(19) United States
(12) Patent Application Publication
(10) Pub. No.: US 2022/0333201 A1
(43) Pub. Date: Oct. 20, 2022

(54) METHOD FOR DETERMINING THE RISK OF OCCURRENCE OF A HEALTHCARE-ASSOCIATED INFECTION IN A PATIENT

(71) Applicants: BIOMÉRIEUX, Marcy L'Etoile (FR); BIOASTER, Lyon (FR); HOSPICES CIVILS DE LYON, Lyon (FR)

(72) Inventors: Laurence VACHOT, Fontaine (FR); François MALLET, Villeurbanne (FR); Guillaume MONNERET, Lyon (FR); Virginie MOUCADEL, Sassenage (FR); Alexandre PACHOT, Sulignat (FR); Estelle PERONNET, Lyon (FR); Thomas RIMMELÉ, Lyon (FR); Julien TEXTORIS, Villeurbanne (FR); Fabienne VENET, Lyon (FR)

(73) Assignees: BIOMÉRIEUX, Marcy L'Etoile (FR); BIOASTER, Lyon (FR); HOSPICES CIVILS DE LYON, Lyon (FR)

(21) Appl. No.: 17/764,323

(22) PCT Filed: Sep. 25, 2020
(86) PCT No.: PCT/FR2020/051673

§ 371 Date: Mar. 28, 2022

(30) Foreign Application Priority Data
Sep. 27, 2019 (FR) 19/10723

(51) Int. Cl. C12Q 1/6883; G16H 50/30

(52) U.S. Cl. CPC C12Q 1/6883; G16H 50/30; C12Q 2600/158

(57) ABSTRACT

An in vitro or ex vivo method for determining the risk of occurrence of a healthcare-associated infection in a patient, including a step of measuring the expression of CX3CR1, in a biological sample of said patient.

The present invention concerns an in vitro or ex vivo method for determining the risk of occurrence of a healthcare-associated infection in a patient, comprising a step for measuring the expression of CX3CR1, in a biological sample of said patient.

The development of healthcare-associated infections is a major complication related to medical care, particularly in medical care structures such as hospitals (where we will more specifically talk about nosocomial infections). It has been demonstrated that nosocomial infections in intensive care units, which occur in 20 to 40% of patients, have been associated with increased morbidity and mortality, a longer duration of need for organ failure supportive care, longer hospital stays, higher healthcare costs, and a considerable use of antibiotics, contributing to antimicrobial resistance. The apparition of healthcare-associated infections has been particularly exacerbated in recent years, due to the increase in multi-resistant pathogens. The World Health Organization (WHO) estimates the number of nosocomial infections in hospitals in Europe at about 5 million, leading to about 50,000 deaths and an additional annual cost of 13 to 24 billion euro. Many factors influence the occurrence and development of healthcare-associated infections, such as the patient's general state of health, but also factors related to patient management (e.g the administration of antibiotics and/or the use of invasive medical devices), factors related to the hospital environment (e.g the ratio of the number of nurses to the number of patients), and the variable use of aseptic techniques by the hospital staff. Recommendations have been published, and the establishment of infection control programs has been encouraged, in particular by the US Department of Health and Human Services, the European Center for Disease Prevention and Control, the WHO and the national agencies, for which the prevention and reduction of healthcare-associated infections have become a major priority. It has been demonstrated that healthcare-associated infection control programs turn out to be particularly effective in reducing severe infections. However, it has been estimated that a maximum of 65 to 70% of cases of blood and urinary tract infections, related to the placement of catheters, and of 55% of cases of pneumonia associated with mechanical ventilation and infections at the surgical site, could be avoided. Moreover, the observance and application of procedures according to the recommendations might be complicated in some hospitals, especially in low- and middle-income countries. The early identification of patients at risk of developing a healthcare-associated infection would be a key step in the prevention of these infections and the management of these patients. According to some models, a biomarker that would reduce the time to identify healthcare-associated infections in a high-risk population would reduce mortality in these patients, with a good cost/effectiveness ratio. However, there is currently no clinical in vitro diagnostic test for identifying patients at high risk of contracting a healthcare-associated infection.

Yet, it has been discovered that, quite surprisingly, the measurement of the expression of the CX3CR1 gene, which encodes for the fractalkine (or CX3CL1) receptor, allows determining the risk of occurrence of a healthcare-associated infection in a patient. The patients at high risk of developing a healthcare-associated infection could advantageously benefit from an immunostimulatory immune treatment or an individualized management. In the literature, a decrease in the expression of CX3CR1 has already proved to be associated with an increase in mortality, in particular in patients in a septic state, and more particularly in a septic shock (Pachot et al. (2008), J Immunol 180: 6421-6429), but the utility of measuring the CX3CR1 expression for prediction of occurrence of healthcare-associated infections has never been demonstrated or suggested.

Thus, an object of the present invention is an in vitro or ex vivo method for determining the risk of occurrence of a healthcare-associated infection in a patient, comprising a step for measuring the expression of CX3CR1 (chromosomal location of the gene according to GRCh38/hg38: chr3:39,263,494-39,281,735), in a biological sample of said patient.

In the context of the present invention:

The term «patient» refers to an individual (human being) who has come into contact with a healthcare professional, such as a doctor (for example, a general practitioner) or a medical structure or a health facility (for example, a hospital, and more particularly the emergency unit, the resuscitation unit, an intensive care unit or an on-going care unit, or a medical structure for the elderly, of the nursing home type). The patient may be, for example, an elderly person, as part of a vaccination protocol (in particular in a nursing home or even with a general practitioner);

An infection is so-called «healthcare-associated», if it occurs during or after a (diagnostic, therapeutic, palliative, preventive, educational or surgical) of a patient by a healthcare professional, and if it has been neither present nor incubating at the start of treatment. Healthcare-associated infections (HAIs) comprise infections developed within a healthcare facility (known as nosocomial infections) but also during healthcare delivered outside this setting. When the infectious state at the start of treatment is not specifically known, a delay of at least 48 hours or a delay greater than the incubation period is commonly accepted to define a HAI. For surgical site infections, infections occurring within 30 days of the surgery or, if an implant, prosthesis or prosthetic material are placed in the year following the surgery, are usually considered to be healthcare-associated. The infection may be of bacterial, fungal or even viral origin. It may also be the reactivation of potentially pathogenic latent viruses, such as TTV or herpes viruses, for example CMV;
The term «biological sample» refers to any sample from a patient, and could be of different natures, such as blood or its derivatives, sputum, urine, stool, skin, cerebrospinal fluid, bronchoalveolar lavage fluid, saliva, gastric secretions, semen, seminal fluid, tears, spinal cord, trigeminal nerve ganglion, adipose tissue, lymphoid tissue, placental tissue, gastrointestinal tract, tissue of the genital tract, tissue of the central nervous system. In particular, this sample may be a biological fluid, such as a blood sample or a sample derived from blood, which may in particular be chosen from whole blood (as collected from the venous route, that is to say containing the white and red cells, platelets and plasma), plasma, serum, as well as any type(s) of cells extracted from blood, such as peripheral blood mononuclear cells (or PBMCs, containing lymphocytes (B, T and NK cells), dendritic cells and monocytes), subpopulations of B cells, purified monocytes, or neutrophils.

Preferably, in the method as described before:
the patient is a patient in a healthcare facility, preferably in a hospital, more preferably in the emergency unit, the resuscitation unit, the intensive care unit or on-going care unit; in a particularly preferred manner, the patient is a patient in a septic state (more particularly, in a septic shock), a patient suffering from burns (more particularly, severe burns), a patient suffering from trauma (more particularly, severe trauma), or a patient undergoing surgery (more particularly, major surgery); and
the method allows determining the risk of occurrence of a nosocomial infection in said patient.

In the case of a patient in a septic state (already suffering from a first infection), the method according to the invention allows determining the risk of occurrence of a secondary infection.

By a septic patient (or patient with sepsis), it should be understood a patient with at least one life-threatening organ failure caused by an inappropriate host response to an infection. By septic shock, it should be understood a sepsis subtype, in which hypotension persists, despite adequate vascular filling.

Preferably, the method according to the invention, as previously described, in all embodiments thereof, allows determining the risk of occurrence of a healthcare-associated infection in a patient:
within 15 days from the day of the immuno-inflammatory attack (i.e. the trauma for patients with trauma, the burn for patients with burns, the surgery for patients having undergine a surgery or the diagnosis of sepsis for septic patients), namely during the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th} or 15^{th}, day from the immuno-inflammatory attack (the 1^{st} day corresponding here to the day of occurrence of the immuno-inflammatory attack); the collection of the biological sample which may have been carried out in particular during the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th} or 15^{th}, day from the immuno-inflammatory attack, preferably during the 1^{st}, 2^{nd}, 3^{rd}, 4^{th}, 5^{th}, 7^{th}, 6^{th} or 7^{th}, day from the immuno-inflammatory attack, more preferably during the 3^{rd}, 4^{th}, 5^{th}, 7^{th}, 6^{th}, or 7^{th}, day from the immuno-inflammatory attack; and/or
within 7 days, within 6 days, within 5 days or within 4 days following the day on which the collection of the biological sample has been performed (regardless of the day on which this collection has been performed), that is to say during the 3^{rd}, 4^{th}, 5^{th}, 7^{th}, 6^{th} or 7^{th}, day following the day on which the collection of the biological sample has been performed (the 1^{st} day herein corresponding to the day after the day on which the collection of the biological sample has been performed).

Preferably, in the method as previously described, in all embodiments thereof, the biological sample is a blood sample, preferably a whole blood sample or a sample derived from blood (e.g. PBMCs, which may be obtained by the Ficoll method, well known to those skilled in the art, or purified monocytes).

Preferably, the method as previously described, in all embodiments thereof, further comprises a step of measuring, in the biological sample of the patient, the expression of another gene of interest, selected from the list consisting of: ADGRE3, BTLA, CD3D, CD74, CD274 (also known as PD-L1), CTLA4 (also known as CD152), HP, ICOS, IFNG, IL1RN, IL6, IL7R (also known as CD127), IL10, IL15, MDC1, PDCD1 (also known as PD-1 and CD279), S100A9, TDRD9 and ZAP70; more preferably, the other gene of interest is selected from the list consisting of: BTLA, CD3D, CD74, CD274, CTLA4, HP, ICOS, IFNG, IL1RN, IL7R, IL10, IL15, PDCD1 and S100A9.

TABLE 1
Chromosomal location of the genes whose expression
may be measured in combination with the measurement
of the expression of CX3CR1

| Biomarker (gene) | Chromosomal location (GRCh38/hg38) |
|---|---|
| ADGRE3 | chr19: 14,619,117-14,690,027 |
| BTLA | chr3: 112,463,966-112,499,702 |
| CD3D | chr11: 118,338,954-118,342,744 |
| CD74 | chr5: 150,400,041-150,412,936 |
| CD274 | chr9: 5,450,381-5,470,567 |
| CTLA4 | chr2: 203,867,771-203,873,965 |
| HP | chr16: 72,054,592-72,061,056 |
| ICOS | chr2: 203,936,731-203,961,579 |
| IFNG | chr12: 68,154,768-68,159,741 |
| IL1RN | chr2: 113,099,365-113,134,016 |
| IL6 | chr7: 22,725,442-22,732,002 |
| IL7R | chr5: 35,852,695-35,879,603 |
| IL10 | chr1: 206,767,602-206,774,607 |
| IL15 | chr4: 141,636,583-141,733,987 |
| MDC1 | chr6: 30,699,807-30,717,966 |
| PDCD1 | chr2: 241,849,881-241,858,908 |
| S100A9 | chr1: 153,357,854-153,361,027 |
| TDRD9 | chr14: 103,928,438-104,052,667 |
| ZAP70 | chr2: 97,713,560-97,744,327 |

The measurement of the expression (or of the expression level) of a gene consists in quantifying at least one expression product of the gene. The expression product of a gene, in the context of the present invention, is any biological molecule resulting from the expression of said gene.

More particularly, the expression product of the gene may be an RNA transcript. By «transcript», it should be understood the RNAs, and in particular the messenger RNAs (mRNAs), resulting from the transcription of the gene. More specifically, the transcripts are RNAs produced by the transcription of a gene followed by the post-transcriptional modifications of the pre-RNA forms. In the context of the present invention, the measurement of the level of expression of one or several RNA transcript(s) of the same gene may be performed. Thus, preferably, in the method as previously described, in all embodiments thereof, the expression of the gene(s) (i.e. the expression of CX3CR1, and optionally of another gene of interest from the previously-indicated list) is measured at the RNA or mRNA transcript level. In the case of an mRNA transcript, the detection may be carried out by a direct method, by any method known to those skilled in the art allowing determining the presence of said transcript in the sample, or by indirect detection of the transcript after transformation of the latter into DNA, or after amplification of said transcript or after amplification of the DNA obtained after transformation of said transcript into DNA. Many methods exist for the detection of nucleic acids (see for example Kricka et al., Clinical Chemistry, 1999, No. 45(4), p. 453-458; Relier G H et al., DNA Probes, 2nd Ed., Stockton Press, 1993, sections 5 and 6, p. 173-249). The expression of the genes may in particular be measured by Reverse Transcription-Polymerase Chain Reaction or RT-PCR, preferably by quantitative RT-PCR or RT-qPCR (for example using the FilmArray® technology), by sequencing (preferably by high throughput sequencing) or by hybridization techniques (for example with hybridization microchips or by techniques of the NanoString® nCounter® type).

The expression product of the gene may also be a protein and/or a polypeptide which is the product of the translation of at least one of the transcripts of said gene. Thus, in the method as previously described, the expression of the gene(s) may also be measured at the protein level. All of the isoforms of the protein(s), expression product(s) of the gene(s), may be measured, alone or in combination, as marker(s) to determine the risk of occurrence of a healthcare-associated infection in a patient. The measurement of the expression of gene(s) at the protein level in a biological sample may be done according to the techniques widely known to those skilled in the art to determine the quantity, or dose, of one or several analyte(s) in a biological sample. As examples, mention may be made of assays by immunoassays, such as ELISA (Enzyme Linked Immuno Sorbent Assay), ELFA (Enzyme Linked Fluorescent Assay) and RIA (Radio Immuno Assay), and assays by mass spectrometry.

The measurement of the expression level of a gene allows determining the quantity of one or several transcript(s) (or one or several protein(s)) present in the biological sample or giving a derived value. A value derived from the quantity may for example be the absolute concentration, calculated thanks to a calibration curve obtained from successive dilutions of a solution of amplicons (or proteins or polypeptides) of known concentration. It may also correspond to the value of the standardized and calibrated quantity, such as the CNRQ (Calibrated Normalized Relative Quantity, (Hellemans et al. (2007), Genome biology 8(2):R19), which integrates the values of a reference sample (or of a calibrator) and one or several housekeeping gene(s) (also called reference genes). Examples of housekeeping genes include the genes DECR1, HPRT1, PPIB, RPLPO, PPIA, GLYR1, RANBP3, 18S, GAPDH and ACTB.

Thus, preferably, in the method as previously described, in all embodiments thereof, the expression of the gene(s) of interest is normalized with respect to the expression of one or several housekeeping gene(s) (or reference genes), as known to those skilled in the art; more preferably using one or more of the following housekeeping genes: DECR1 (chromosomal location of the gene according to GRCh38/hg38: chr8:90,001,352-90,053,633), HPRT1 (chromosomal location of the gene according to GRCh38/hg38: chrX:134,452,842-134,520,513) and PPIB (chromosomal location of the gene according to GRCh38/hg38: chr15:64,155,812-64,163,205).

Preferably, in the method as previously described, in all embodiments thereof, the expression of the gene(s) of interest (preferably, the normalized expression) in the patient's biological sample is compared to a reference value or to the expression of the same gene(s) of interest (preferably, the normalized expression) in a biological reference sample (these data being used for the calculation of the CNRQ, as mentioned above). The reference sample may be for example a sample from a volunteer (healthy individual), from a patient, or a mixture of samples from several volunteers (on the one hand) or from several patients (on the other hand). The reference sample may also be a sample taken from a volunteer (or a mixture of samples taken from several volunteers) then treated ex vivo with an immune system stimulating agent (such as LPS or lipopolysaccharide). The reference sample may also be a mixture of untreated sample(s) and sample(s) treated ex vivo with an immune system stimulating agent.

Preferably, the method for determining the risk of occurrence of a healthcare-associated infection, as previously described, in all embodiments thereof, also comprises a healthcare management step to reduce the risk of occurrence of a healthcare-associated infection. A patient identified as being at increased risk of developing a healthcare-associated infection may have appropriate healthcare management with the aim of reducing the risk of developing a healthcare-associated infection and, for example, to reduce the risk of developing sepsis, septic shock or the risk of death. Examples of healthcare management include an immunomodulatory treatment adapted to the patient or a prophylactic antibiotic treatment, the two treatments may be combined and/or refer to an on-going care unit or resuscitation unit in order to reduce the risk of occurrence of a healthcare-associated infection, for example reducing the risk of developing sepsis, septic shock or even the risk of death in the days following the measurement of the expression of the biomarker(s). Preferably, the immunomodulatory treatment is an immunostimulatory treatment, if the individual is determined to have an immunosuppressed status, or an anti-inflammatory treatment, if the individual is determined to have an inflammatory status. Among the immunostimulatory treatments which may be selected, mention may be made for examples of the group of interleukins, in particular IL-7, IL-15 or IL-3, growth factors, in particular GM-CSF, interferons, in particular IFNγ, Toll agonists, antibodies, in particular anti-PD1, anti-PDL1, anti-LAG3, anti-TIM3, anti-IL-10 or anti-CTLA4 antibodies, transferrins and molecules that inhibit apoptosis, FLT3L, Thymosin al, adrenergic antagonists. Among the anti-inflammatory treatments, mention may be made in particular of the group of glucocorticoids, cytostatic agents, molecules acting on immunophilins and cytokines, molecules blocking the IL-1 receptor and anti-TNF treatments. Examples of appropriate prophylactic antibiotic treatments to prevent pneumonia are described in particular in “Annales Françaises d'Anesthesie et de Reanimation” (30; 2011; 168-190). Conversely, a patient who does not present a risk of occurrence of a healthcare-associated infection may be quickly transferred to a daytime hospital service, for example an infectiology service, rather than remaining in a service with close monitoring which he won't need.

Another object of the invention is a kit comprising means for amplifying and/or means for detecting the expression (preferably primers and/or probes, or antibodies) of CX3CR1 and of another gene, selected from the list consisting of: ADGRE3, BTLA, CD3D, CD74, CD274, CTLA4, HP, ICOS, IFNG, URN, IL6, IL7R, IL10, IL15, MDC1, PDCD1, S100A9, TDRD9 and ZAP70 (preferably, the another gene is selected from the list consisting of: BTLA, CD3D, CD74, CD274, CTLA4, HP, ICOS, IFNG, URN, IL7R, IL10, IL15, PDCD1 and S100A9 or from the list consisting of: ADGRE3, BTLA, CD3D, CD74, CD274, CTLA4, HP, IFNG, IURN, IL6, IL7R, IL10, MDC1, PDCD1, S100A9, TDRD9 and ZAP70; more preferably from the list consisting of: BTLA, CD3D, CD74, CD274, CTLA4, HP, IFNG, URN, IL7R, IL10, PDCD1 and S100A9); said kit being characterized in that all of the amplification and/or detection means of said kit allow the detection and/or amplification of at most 100, preferably at most 90, preferably at most 80, of preferably at most 70, preferably at most 60, preferably at most 50, preferably at most 40, preferably at most 30, preferably at most 20, preferably at most 10, preferably at most 5 biomarkers, preferably at most 4, preferably at most 3, preferably at most 2, in total. By «biomarker» (or «marker») it should be understood an objectively measurable biological characteristic that represents an indicator of normal or pathological biological processes or of pharmacological response to a therapeutic intervention. This biomarker may in particular be detectable at the mRNA or protein level. More particularly, the biomarker may be an endogenous biomarker or loci (such as a gene or a HERV/Human Endogenous Retro Virus, which are found in the chromosomal material of an individual) or an exogenous biomarker (such as a virus).

Thus, said kit may for example also comprise means for amplifying and/or detecting one or several housekeeping gene(s) (preferably selected from the list consisting of: DECR1, HPRT1 and PPIB). The kit may also comprise positive control means allowing assessing the quality of the RNA extraction, the quality of any amplification and/or hybridization process.

By «primer» or «amplification primer», it should be understood a nucleotide fragment which may consist of 5 to 100 nucleotides, preferably of 15 to 30 nucleotides, and possessing a specificity of hybridization with a target nucleotide sequence, under conditions determined for the initiation of an enzymatic polymerization, for example in an enzymatic amplification reaction of the target nucleotide sequence. In general, “pairs of primers”, consisting of two primers, are used. When it is desired to carry out the amplification of several different biomarkers (e.g. genes), several different pairs of primers are preferably used, each preferably having the ability to hybridize specifically with a different biomarker.

By «probe» or «hybridization probe», is should be understood a nucleotide fragment typically consisting of 5 to 100 nucleotides, preferably of 15 to 90 nucleotides, even more preferably of 15 to 35 nucleotides, possessing a hybridization specificity under determined conditions to form a hybridization complex with a target nucleotide sequence. The probe also includes a reporter (such as a fluorophore, an enzyme or any other detection system), which will allow the detection of the target nucleotide sequence. In the present invention, the target nucleotide sequence may be a nucleotide sequence comprised in a messenger RNA (mRNA) or a nucleotide sequence comprised in a complementary DNA (cDNA) obtained by reverse transcription of said mRNA. When it is desired to target several different biomarkers (e.g. genes), several different probes are preferably used, each preferably having the ability to hybridize specifically with a different biomarker.

By «hybridization», it should be understood the process during which, under appropriate conditions, two nucleotide fragments, such as for example a hybridization probe and a target nucleotide fragment, having sufficiently complementary sequences, are capable of forming a double strand with stable and specific hydrogen bonds. A nucleotide fragment «capable of hybridizing» with a polynucleotide is a fragment capable of hybridizing with said polynucleotide under hybridization conditions, which may be determined in each case in a known manner. The hybridization conditions are determined by the stringency, that is to say the rigor of the operating conditions. The hybridization is even more specific as it is performed at higher stringency. The stringency is defined in particular according to the base composition of a probe/target duplex, as well as by the degree of mismatch between two nucleic acids. The stringency can also be a function of the reaction parameters, such as the concentration and the type of ionic species present in the hybridization solution, the nature and the concentration of denaturing agents and/or the hybridization temperature. The stringency of the conditions under which a hybridization reaction is to be performed will depend primarily on the used hybridization probes. All of these data are well known and the appropriate conditions may be determined by one skilled in the art. In general, depending on the length of the used hybridization probes, the temperature for the hybridization reaction is comprised between about 20 and 70° C., particularly between 35 and 65° C. in a saline solution at a concentration of about 0.5 to 1 M. A step of detecting the hybridization reaction is then carried out.
By «enzymatic amplification reaction», it should be understood a process generating multiple copies of a target nucleotide fragment, by the action of at least one enzyme. Such amplification reactions are well known to one skilled in the art and the following techniques may be mentioned in particular: PCR (Polymerase Chain Reaction), LCR (Ligase Chain Reaction), RCR (Repair Chain Reaction), 3SR (Self Sustained Sequence Replication) with the patent application WO-A-90/06995, NASBA (Nucleic Acid Sequence-Based Amplification), TMA (Transcription Mediated Amplification) with U.S. Pat. No. 5,399,491, and LAMP (Loop mediated isothermal amplification) with the U.S. Pat. No. 6,410,278. When the enzymatic amplification reaction is a PCR, we will talk more particularly of RT-PCR (RT standing for «reverse transcription»), when the amplification step is preceded by a messenger RNA reverse-transcription step (mRNA) into complementary DNA (cDNA), and from qPCR or RT-qPCR when PCR is quantitative.

Another object of the invention is the use:
of means for amplifying and/or means for detecting the expression (preferably primers and/or probes, or antibodies) of CX3CR1, and optionally also of another gene, selected from the list consisting of: ADGRE3, BTLA, CD3D, CD74, CD274, CTLA4, HP, ICOS, IFNG, URN, IL6, IL7R, IL10, IL15, MDC1, PDCD1, S100A9, TDRD9 and ZAP70 (preferably the other gene is selected from the list consisting of: BTLA, CD3D, CD74, CD274, CTLA4, HP, ICOS, IFNG, IL1 RN, IL7R, IL10, IL15, PDCD1 and S100A9 or in the list consisting of: ADGRE3, BTLA, CD3D, CD74, CD274, CTLA4, HP, IFNG, URN, IL6, IL7R, IL10, MDC1, PDCD1, S100A9, TDRD9 and ZAP70; more preferably from the list consisting of: BTLA, CD3D, CD74, CD274, CTLA4, HP, IFNG, IL1 RN, IL7R, IL10, PDCD1 and S100A9); or
of a kit comprising such amplification and/or detection means, and preferably all of the amplification and/or detection means of said kit allow the detection and/or amplification of at most 100, preferably at most 90, preferably at most 80, preferably at most 70, preferably at most 60, preferably at most 50, preferably at most 40, preferably at most 30, preferably at most 20, preferably at most 10, preferably at most 5 biomarkers, preferably at most 4, preferably at most 3, preferably at most 2 biomarkers, in total, and optionally said kit comprises means for amplifying and/or detecting the expression of one or several housekeeping gene(s) (preferably selected from the list consisting of: DECR1, HPRT1 and PPIB),

to determine the risk of occurrence of a healthcare-associated infection, preferably a nosocomial infection, in a patient, preferably a patient within a healthcare facility, more preferably within a hospital, preferably in the emergency unit, the resuscitation unit, the intensive care unit or the on-going care unit; particularly preferably, the patient is a patient in a septic state (more particularly in septic shock), a patient suffering from burns (more particularly, severe burns), a patient suffering from trauma (more particularly, severe trauma), or a patient undergoing surgery (more particularly, major surgery).

The present invention is illustrated without limitation by the following examples.

### EXAMPLE 1: THE MEASUREMENT OF THE EXPRESSION OF CX3CR1 ALLOWS PREDICTING THE RISK OF OCCURRENCE OF A HEALTHCARE-ASSOCIATED INFECTION IN A PATIENT

Materials and Methods

A prospective, longitudinal and monocentric observational clinical study has been carried out at the Edouard Herriot Hospital (Lyon, France). The design of this clinical study has been published in Rol et al. (2017), BMJ Open 7(6): e015734. The clinical study was approved by the National Agency for the Safety of Medicines and Health Products (ANSM) in November 2015 and the South-East II Personal Protection Committee in December 2015. Amendments to the protocol were made in July 2016, then in January 2017. In brief, a total of 377 patients, in a septic state (n=35) or in septic shock (n=72), suffering from severe burns (n=24), severe trauma (n=137) or hospitalized in a resuscitation unit or intensive care unit after major surgery (n=109), and 175 healthy volunteers have been included between December 2015 and March 2018.
Patients in septic state/in septic shock: according to the first clinical protocol, only patients in septic shock have been included, on the basis of a suspicion of an infectious focus, a start of treatment with catecholamines within 48 hours following admission to the resuscitation unit and of treatment with catecholamines (noradrenaline)>0.25 μg/kg/min for at least 2 hours. Then, the eligibility criteria were modified in August 2016, following the publication of a new definition of septic shock, Sepsis 3 (Singer et al. (2016), JAMA 315(8):801-810). The patients in septic shock have therefore been included on the basis of a suspicion of an infectious focus, a start of treatment with catecholamines within 48 hours following admission to resuscitation unit and of vasopressor therapy necessary to maintain blood pressure 65 mm Hg and lactate concentration >2 mmol/L (18 mg/dL), despite the correction of hypovolaemia. In 2017, the possibility was added to include patients in a sepsis state (according to the Sepsis 3 definition), namely the suspicion of an infectious focus and the increase in the SOFA score 2 points compared to the basic SOFA within 48 hours following admission to the resuscitation unit. For this population, day 1 corresponds to the day of diagnosis of sepsis or septic shock;
Severe trauma: in the first protocol, only patients with severe trauma have been included (Injury Severity Score (ISS) 25). In August 2016, the possibility was added to also include less severe injuries (16<ISS<24). For this population, day 1 corresponds to the day of admission to the resuscitation unit or intensive care unit (trauma day);
Major surgery: in the first protocol, only esogastrectomy, Bricker-type bladder resection, cephalic pancreaticoduodenectomy and surgery of the abdominal aorta by laparotomy have been considered. Other types of surgery with a high risk of complication were added in January 2017: (total or caudal) pancreatectomy, neuroendocrine tumors, hepatectomy (on the right side), extended colectomy (laparotomy), abdoperineal resection, nephrectomy (laparotomy, PKD), ilio-femoral bypass (Scarpa). For this population, day 1 corresponds to the day of surgery;
Severe burns: the patients have been selected on the basis of a total surface area of burns greater than 30%. For this population, day 1 corresponds to the day of admission to the resuscitation unit or intensive care unit (˜day of the burn).

The exclusion criteria have mainly related to factors that could have impacted the immune status and biased the results (for example: severe neutropenia, corticosteroid treatments, onco-haematological pathology, etc.). Each event leading to a suspected healthcare-associated infection occurring in the hospital before day 30 has been independently reviewed by three physicians not involved in the recruitment of the patients. Twenty-six percent of the patients have developed at least one healthcare-associated infection before day 30 or before leaving the hospital.

Blood samples have been collected in PAXgene® tubes (ref. 762165, PreAnalytiX GmbH Hombrechtikon Switzerland), once for the healthy volunteers, and several times for the patients, i.e. 3-4 times the first week (on days 1 or 2: D1/2, on days 3 or 4: D3/4 and on days 5, 6 or 7: D5/7), then 3 times later on (around D14, D28 and D60).

The expression level of CX3CR1 has been measured in these samples by RT-qPCR. RNA has been extracted from whole blood samples using the Maxwell HT Simply RNA kit (ref. AX2420, Promega) and the EVO automated platform (TECAN), following the kit supplier's instructions. Then, 10 ng of total RNA has been reverse-transcribed into complementary DNA (cDNA), using the Fluidigm Reverse transcription master mix (ref. PN100-6472 A1, Fluidigm), following the supplier's instructions. The expression of CX3CR1 has then been quantified by qPCR, using Fluidigm's Biomark HD real-time PCR system.

First, a cDNA-preamplification step has been performed according to the supplier's recommendations using the PreAmp master mix (Ref. PN100-5876 B1, Fluidigm). Then, a 1/5 dilution of the preamplified cDNAs has been made and the qPCRs have been performed on fluidics integrated circuits 192.24 (Ref. PN100-6170 C1), as recommended by the supplier. The references of the probes and primers used for the qPCR are presented in Table 2.

The threshold cycles (or Ct) have then been determined. The normalization of the expression of CX3CR1 in CNRQ (Calibrated Normalized Relative Quantity) has been carried out using the geometric mean of the Ct of 3 housekeeping genes (DECR1, HPRT1, PPIB) and a calibrator (corresponding to a mixture of treated samples ex vivo by LPS (immune system stimulating agent) (50%) and untreated samples ex vivo (50%), from healthy volunteers/individuals) in each fluidics integrated circuit 192.24, as described in Hellemans et al. (2007), Genome biology 8(2):R19.

TABLE 2
Probes and primers used for qPCR

| Biomarker | | Reference of the supplier of |
|---|---|---|
| (gene) | Type | the used probes and primers |
| CX3CR1 | Gene of interest | Hs04971470_s1 (ThermoFisher) |
| DECR1 | Gene of interest | Hs00154728_m1 (ThermoFisher) |
| HPRT1 | Gene of interest | Hs99999909_m1 (ThermoFisher) |
| PPIB | Gene of interest | Hs01018503_m1 (ThermoFisher) |

Regarding the data analysis, the associations between the expression of CX3CR1, measured at different time points during the first week, and the occurrence of a healthcare-associated infection before day 30 from inclusion in the study have been assessed. The results have been calculated in the form of Hazard Ratios expressed as inter-quartile distance with the associated 95% confidence interval (HR IQR). Then, univariate logistic regressions have been implemented to predict the risk of occurrence of a healthcare-associated infection before day 15. The power of the values predicted by the logistic regression to discriminate between healthcare-associated infection and absence of a healthcare-associated infection has been quantified by the area under the curve (AUC) of the ROC (Receiver Operating Characteristic) curve, and 95% confidence intervals have been estimated.

Then, the association between the expression of CX3CR1 and the occurrence of a healthcare-associated infection has been assessed for different time intervals from the occurrence of the infection (i.e. time period between the sample collection and the 1st occurrence of the infection). The considered different time periods have been: a healthcare-associated infection within 4 days and within 7 days after the sample collection, regardless of when the sample has been collected. For each patient who has developed a healthcare-associated infection, the considered sample corresponds to the closest sample collection before the occurrence of the first episode of a healthcare-associated infection.

For the patients who have not developed a healthcare-associated infection (i.e. control patients), a matching method has been used to select, for each case, a control patient with the same sample collection day, and close SOFA and Charlson scores. Finally, a unique control has been selected for each unique case. Univariate logistic regressions have been implemented. The power of the values predicted by logistic regression to discriminate between healthcare-associated infection and absence of a healthcare-associated infection has been quantified by the area under the curve (AUC) of the ROC curve, and 95% confidence intervals have been estimated.

Results

A decrease in the expression of CX3CR1 at the mRNA level, measured at D3/4 or D5/7 from the inclusion in the cohort, is associated with a higher risk of occurrence of a healthcare-associated infection before D30 (D3/4: HR IQR=0.54 [0.39-0.74], p=0.0001; D5/7: HR IQR=0.57 [0.42-0.79], p=0.0006) in the overall population of the patients. This association has always been significant, for the two measurement times of the expression of CX3CR1, after adjustment with the SOFA and Charlson scores (D3/4: HR IQR=0.61 [0.44-0.85], p=0.003; D5/7: HR IQR=0.65 [0.46-0.91], p=0.01).

Moreover, the prediction models have shown that the expression of CX3CR1 at the mRNA level, measured on D3/4 or on D5/7 from the inclusion in the cohort, have allowed the occurrence of a healthcare-associated infection before day 15 from the inclusion in the cohort (Table 3).

TABLE 3
Performance (AUC and 95% confidence interval, CI) of the
measurement of the expression of CX3CR1, measured on D
3/4 or on D 5/7 from the inclusion in the cohort, for the
prediction of occurrence of a healthcare-associated infection
before day 15 from the inclusion in the cohort.

| Day of the collection of the sample | AUC (CI) | |
|---|---|---|
| D 3/4 | 0.677 | (0.571-0.783) |
| D 5/7 | 0.758 | (0.655-0.86) |

The prediction models have also shown that the expression of CX3CR1 at the mRNA level, measured on D3/4 or on D5/7, has allowed predicting the occurrence of a healthcare-associated infection within 4 days or within 7 days following the collection of the sample (Table 4).

TABLE 4
Performance (AUC and 95% confidence interval, CI) of the measurement
of the expression of CX3CR1, for the prediction of the occurrence
of a healthcare-associated infection within 4 days or within
7 days following the sample collection.

| Time interval between the collection | |
|---|---|
| of the sample and the possible | |
| occurrence of the first healthcare- | |
| associated infection | AUC (CI) |
| 4 days | 0.642 (0.542-0.742) |
| 7 days | 0.657 (0.567-0.746) |

Thus, the obtained results show that the measurement of the expression of CX3CR1 alone allows predicting the occurrence of healthcare-associated infection(s) within 15 days from the immuno-inflammatory attack, within 4 days following the collection of the sample or within 7 days of the collection of the sample.

### EXAMPLE 2: THE MEASUREMENT OF THE EXPRESSION OF CX3CR1 AND OF ANOTHER GENE ALLOWS IMPROVING THE PREDICTIVE PERFORMANCE OF THE RISK OF OCCURRENCE OF A HEALTHCARE-ASSOCIATED INFECTION

Materials and Methods

The Materials and Methods are identical to those of Example 1, except that 1) the cDNA preamplification step has been carried out with another PreAmp master mix reference (Ref. PN100-5875 C1, Fluidigm) for certain genes (BTLA and IL15, as well as the reference genes which have been measured with the 2 references) and followed by an additional step of treatment with exonuclease I (ref. PN100-5875 C1, Fluidigm), and that in addition for these genes, the amplification step has been carried out with another type of fluidics integrated circuits 192.24 (Ref. PN100-7222 C1), as recommended by the supplier, and 2) that herein, multivariate logistic regressions (combination of the measurement of the expression of CX3CR1 and of another gene, selected from the list consisting of: BTLA, CD3D, CD74, CD274, CTLA4, HP, ICOS, IFNG, IL1 RN, IL7R, IL10, IL15, PDCD1 and S100A9) have been performed.

Thus, the expression of the genes of interest (with the exception of BTLA and IL15) has been measured in TaqMan chemistry, with a normalization using the expression of the reference genes also measured in TaqMan chemistry (ThermoFisher reference, including two primers and a probe). The expression of the BTLA and IL15 genes has been measured using SYBR Green chemistry, with normalization using the expression of the reference genes also measured using SYBR Green chemistry (Integrated DNA Technologies reference, including two primers). In addition to the probes and primers already presented in Table 2 (for CX3CR1 and the reference genes in TaqMan chemistry), the additional probes and primers used in this example are presented in Table 5 (for the other genes of interest and the reference genes in SYBR Green chemistry).

TABLE 5
Additional probes and primers used for qPCR

| | | Supplier's reference or sequences |
|---|---|---|
| Biomarker | | corresponding to the used probes |
| (gene) | Type | and primers |
| BTLA | Gene of interest | Hs.PT.58.14525368 (Integrated DNA |
| | | Technologies) |
| CD3D | Gene of interest | Hs00174158_m1 (ThermoFisher) |
| CD74 | Gene of interest | Hs00959493_g1 (ThermoFisher) |
| CD274 | Gene of interest | Hs01125301_m1 (ThermoFisher) |
| CTLA4 | Gene of interest | Hs00175480_m1 (ThermoFisher) |
| HP | Gene of interest | Hs00605928_g1 (ThermoFisher) |
| ICOS | Gene of interest | Hs04261471_m1 (ThermoFisher) |
| IFNG | Gene of interest | Hs00174143_m1 (ThermoFisher) |
| IL1RN | Gene of interest | Hs00893626_m1 (ThermoFisher) |
| IL7R | Gene of interest | primer (forward) : |
| | | CTCTGTCGCTCTGTTGGTC |
| | | primer (reverse) : |
| | | TCCAGAGTCTTCTTATGATCG |
| | | Probe: CTATCGTATGGCCCAGTCTCC |
| IL10 | Gene of interest | Hs00961620_g1 (ThermoFisher) |
| | Gene of interest | Hs.PT.58.21299580 (Integrated DNA |
| IL15 | | Technologies) |
| PDCD1 | Gene of interest | Hs01550088_m1 (ThermoFisher) |
| S100A9 | Gene of interest | Hs00610058_m1 (ThermoFisher) |
| DECR1 | Gene of interest | Hs.PT.58.19871222 (Integrated DNA |
| | | Technologies) |
| HPRT1 | Gene of interest | Hs.PT.58v.45621572 (Integrated DNA |
| | | Technologies) |
| PPIB | Gene of interest | Hs.PT.58v.45621572 (Integrated DNA |
| | | Technologies) |

Results

The measurement of the expression of one of these other genes of interest, in addition to the measurement of the expression of CX3CR1, allows improving the predictive performance of the risk of occurrence of a healthcare-associated infection, whether before day 15 from the inclusion in the cohort (Table 6) or within 4 days or within 7 days following the collection of the sample (Table 7).

TABLE 6
Performance (AUC and 95% confidence interval, CI) of the measurement
of the expression of CX3CR1, in combination with another
biomarker (multivariate analysis), measured on D 3/4 or on
D 5/7 from the inclusion in the cohort, for the prediction
of the occurrence of a healthcare-associated infection before
day 15 from the inclusion in the cohort.

| Day of collection | | | |
|---|---|---|---|
| of the sample | Biomarkers | AUC (CI) | |
| D 3/4 | CX3CR1 + BTLA | 0.681 | (0.576-0.785) |
| D 3/4 | CX3CR1 + ICOS | 0.681 | (0.578-0.784) |
| D 3/4 | CX3CR1 + IFNG | 0.683 | (0.58-0.787) |
| D 3/4 | CX3CR1 + IL15 | 0.684 | (0.582-0.785) |
| D 3/4 | CX3CR1 + PDCD1 | 0.693 | (0.587-0.799) |
| D 3/4 | CX3CR1 + HP | 0.693 | (0.589-0.797) |
| D 3/4 | CX3CR1 + CD74 | 0.694 | (0.591-0.797) |
| D 3/4 | CX3CR1 + IL10 | 0.725 | (0.625-0.825) |
| D 3/4 | CX3CR1 + S100A9 | 0.729 | (0.615-0.844) |
| D 5/7 | CX3CR1 + IFNG | 0.759 | (0.655-0.862) |
| D 5/7 | CX3CR1 + CD274 | 0.761 | (0.66-0.863) |
| D 5/7 | CX3CR1 + PDCD1 | 0.763 | (0.667-0.86) |
| D 5/7 | CX3CR1 + CTLA4 | 0.778 | (0.674-0.883) |
| D 5/7 | CX3CR1 + HP | 0.784 | (0.69-0.878) |
| D 5/7 | CX3CR1 + CD74 | 0.8 | (0.708-0.892) |
| D 5/7 | CX3CR1 + ICOS | 0.802 | (0.707-0.897) |
| D 5/7 | CX3CR1 + CD3D | 0.802 | (0.708-0.896) |
| D 5/7 | CX3CR1 + BTLA | 0.803 | (0.701-0.905) |
| D 5/7 | CX3CR1 + S100A9 | 0.803 | (0.702-0.904) |
| D 5/7 | CX3CR1 + IL7R | 0.803 | (0.718-0.888) |
| D 5/7 | CX3CR1 + IL1RN | 0.819 | (0.726-0.911) |
| D 5/7 | CX3CR1 + IL10 | 0.852 | (0.775-0.928) |

TABLE 7
Performance (AUC and 95% confidence interval, CI) of the measurement
of the expression of CX3CR1, in combination with another biomarker
(multivariate analysis), for the prediction of the occurrence
of a healthcare-associated infection within 4 days or within
7 days following the sample collection.

| Time interval between | | | |
|---|---|---|---|
| the collection of the sample | | | |
| and the possible occurrence | | | |
| of the first healthcare- | | | |
| associated infection | Biomarkers | AUC (CI) | |
| 4 days | CX3CR1 + CD3D | 0.648 | (0.549-0.747) |
| 4 days | CX3CR1 + IL15 | 0.653 | (0.545-0.761) |
| 4 days | CX3CR1 + ICOS | 0.654 | (0.555-0.752) |
| 4 days | CX3CR1 + S100A9 | 0.661 | (0.563-0.759) |
| 4 days | CX3CR1 + CD74 | 0.663 | (0.566-0.761) |
| 4 days | CX3CR1 + IFNG | 0.671 | (0.572-0.771) |
| 4 days | CX3CR1 + BTLA | 0.679 | (0.582-0.776) |
| 4 days | CX3CR1 + HP | 0.704 | (0.61-0.798) |
| 7 days | CX3CR1 + CD274 | 0.664 | (0.573-0.754) |
| 7 days | CX3CR1 + CD3D | 0.667 | (0.579-0.756) |
| 7 days | CX3CR1 + IL15 | 0.668 | (0.573-0.762) |
| 7 days | CX3CR1 + IL10 | 0.668 | (0.576-0.76) |
| 7 days | CX3CR1 + IL7R | 0.668 | (0.579-0.756) |
| 7 days | CX3CR1 + IL1RN | 0.671 | (0.581-0.762) |
| 7 days | CX3CR1 + CTLA4 | 0.671 | (0.582-0.759) |
| 7 days | CX3CR1 + IFNG | 0.676 | (0.586-0.765) |
| 7 days | CX3CR1 + ICOS | 0.686 | (0.599-0.773) |
| 7 days | CX3CR1 + BTLA | 0.692 | (0.605-0.779) |
| 7 days | CX3CR1 + CD74 | 0.694 | (0.608-0.78) |
| 7 days | CX3CR1 + S100A9 | 0.699 | (0.614-0.785) |
| 7 days | CX3CR1 + HP | 0.707 | (0.622-0.792) |

1. An in vitro or ex vivo method for determining the risk of occurrence of a healthcare-associated infection in a patient, comprising a step of measuring the expression of CX3CR1, in a biological sample from said patient.

2. The method according to claim 1, wherein the patient is a patient within a healthcare facility and the method allows determining the risk of occurrence of a nosocomial infection in said patient.

3. The method according to claim 1, wherein the patient is a patient within a hospital.

4. The method according to claim 1, wherein it allows determining the risk of occurrence of a healthcare-associated infection in the patient within 15 days from the immuno-inflammatory attack and/or within 7 days following the day when the biological sample collection has been performed.

5. The method according to claim 1, wherein the biological sample is a blood sample.

6. The method according to claim 1, wherein the biological sample is a whole blood sample.

7. The method according to claim 1, wherein it further comprises a step of measuring, in the biological sample of the patient, the expression of another gene, selected from the list consisting of: ADGRE3, BTLA, CD3D, CD74, CD274, CTLA4, HP, ICOS, IFNG, IL1RN, IL6, IL7R, IL10, IL15, MDC1, PDCD1, S100A9, TDRD9 and ZAP70.

8. The method according to claim 1, wherein the expression is measured at the mRNA or protein level.

9. The method according to claim 1, wherein the expression is measured at the mRNA level.

10. The method according to claim 1, wherein the expression is measured by RT-PCR.

11. The method according to claim 1, wherein the expression is measured by sequencing.

12. The method according to claim 1, wherein the expression is measured by hybridization.

13. The method according to claim 9, wherein the expression is normalized with respect to the expression of one or several housekeeping genes.

14. A kit comprising means for amplifying and/or means for detecting the expression of CX3CR1 and of another gene, selected from the list consisting of ADGRE3, BTLA, CD3D, CD74, CD274, CTLA4, HP, IFNG, IL1RN, IL6, IL7R, IL10, MDC1, PDCD1, S100A9, TDRD9 and ZAP70; wherein all of the amplification and/or detection means of said kit allow the detection and/or amplification of at most 100 biomarkers in total.

15. (canceled)
